(19) United States
(12) Patent Application Publication
(10) Pub. No.: US 2008/0091165 A1
(43) Pub. Date: Apr. 17, 2008

(54) Method and apparatus for collecting colostomy waste

(76) Inventor: Richard M. Kushner, Larchmont, NY (US)

Correspondence Address: MORGAN LEWIS & BOCKIUS LLP, 1111 PENNSYLVANIA AVENUE NW, WASHINGTON, DC, 20004, US

(21) Appl. No.: 11/581,041

(22) Filed: Oct. 11, 2006

(51) Int. Cl. A61M 31/00

(52) U.S. Cl. 604/500

(57) ABSTRACT

An improved colostomy waste removal method and apparatus which comprises an adhesive pad adhered to a patient's stomach. The pad contains a generally central hole which fits over the stoma and through which a severed ileum protrudes. The pad includes a hole sized to receive the patient's ileum. The pad bears a removable waste receiving vehicle, e.g., bag into which excrement flows from the stomach via the small intestine and ileum. The pad is adapted to be replaced from time to time and the patient must make a hole through which the ileum must pass. To this end, a pre-shaped cutting tool with a selectable one of a number of hole sizes is employed to readily make the requisite size hole in the pad.

This invention relates to colostomy medical methodology and apparatus and, more specifically, to methods and apparatus for facilitating body waste removal and collection via a transcutaneously projecting ileum while minimizing skin attack from leakage of such waste.

### BACKGROUND OF THE INVENTION

There are medical conditions where the small intestine must be separated from its junction with the large intestine (“ileostomy”). This is done, for example, when the large intestine is infected (colitis), ulcerated, or contains a malignancy. Isolating the large intestine permits its rest and healing by temporarily removing that intestine from the path of body waste flow. The severed, lower end of the small intestine most remote from the stomach (“ileum”), can then be surgically reconnected to the large intestine when possible. There are conditions where the colon is surgically removed—and thus no reconnection is possible. The ileostomy is therefore permanent.

In either event, when the ileum is separated from the large intestine (colon) temporarily or permanently, the body's liquid and solid and semi-solid excrement continues to be generated and must be discharged to an external collection bag or the like. To this end, a hole (“stoma”) is made in the patient's abdomen, and the small intestine's severed ileum passes through the stoma and is retained in place by sutures. An adhesive pad bearing a waste collecting emptyable bag and an ileum receiving, generally central aperture is placed over the stoma and the ileum.

Solid and liquid waste pass from the stomach through the small intestine, flow from the body via the stoma and ileum, and are collected in the bag. This colostomy waste removal can occur over weeks or months, or indefinitely.

There are difficulties with prior art implementations of this-colostomy-mandated procedure. The adhesive pad must be replaced, e.g., once the every several days. When a new pad is applied, the patient (already weakened and distressed) must use a scissors or the like to cut an ileum receiving aperture in the pad which closely conforms to the then-obtaining size of the ileum. The ileum typically changes size during treatment, e.g., reducing in circumference as it heals from the severing surgery and sutures, or increasing its size if there is infection or irritation.

However, the waste effluent passing outward from the ileum contains harsh, caustic and enzymatic materials. These cause irritation or infection if they reach the abdomen or the stoma—as when the hole the patient cuts in the adhesive pad is not a good fit to, but not a restriction of the ileum. A template with different size holes is sometimes provided—but is not an adequate or satisfactory solution to the problem.

It is an object of the present invention to provide improved colostomy methodology and apparatus.

More specifically, it is an object of the present invention to provide improved methods and apparatus for readily effecting and facilitating a properly sized aperture in an ileum receiving replaceable pad.

### SUMMARY OF THE INVENTION

The above and other objects of the present invention are realized in a specific illustrative improved colostomy waste removal method and apparatus which comprises an adhesive pad adhered to a patient's stomach. The pad contains a generally central hole which fits over the stoma and through which a severed ileum protrudes. The pad includes a hole sized to receive the patient's ileum. The pad bears a removable waste receiving vehicle, e.g., bag, into which excrement flows from the stomach via the small intestine and ileum.

The pad is adapted to be replaced from time to time and the patient must make a hole through which the ileum must pass. To this end, a pre-shaped cutting tool with a selectable one of a number of hole sizes is employed to readily make the requisite size hole in the pad. The cutting tool can comprise, for example, a punch with replaceable dies of varying size, or one of plural hand actuated rotatable cutting dies. A regular hole can thus be formed in the pad to conform to the then-obtaining size of the protruding ileum. By “conform” I mean be of a size just ample to pass the ileum in its current state, without any substantial gap between the ileum and inner edge of the hole, thus greatly reducing the prospect of stomach attack or ulceration by the waste products. This also reduces requirement for a second or further operation if the stoma must be moved because of abdomen or stoma compromise at the former stoma area.

### BRIEF DESCRIPTION OF THE DRAWINGS

The present invention will become more clear from the following detailed description thereof, presented in conjunction with the accompanying drawing in which:

FIG. 1 is a depiction of human anatomy from roughly the stomach toward the rectum with an expanded view of the ileum;

FIG. 2 is a plan view of colostomy apparatus of the prior art;

FIG. 3 is a cross-sectional view of colostomy apparatus of the present invention;

FIG. 4a represent first mechanically implemented apparatus for forming a central aperture in the body adhering pad of the present invention.

FIG. 5 is an actuator handle for the die(s) of FIG. 4.

FIGS. 6 and 7 are second and third illustrative mechanically implemented apparatus for forming a central aperture in the body adhering pad of the present invention.

### DETAILED DESCRIPTION

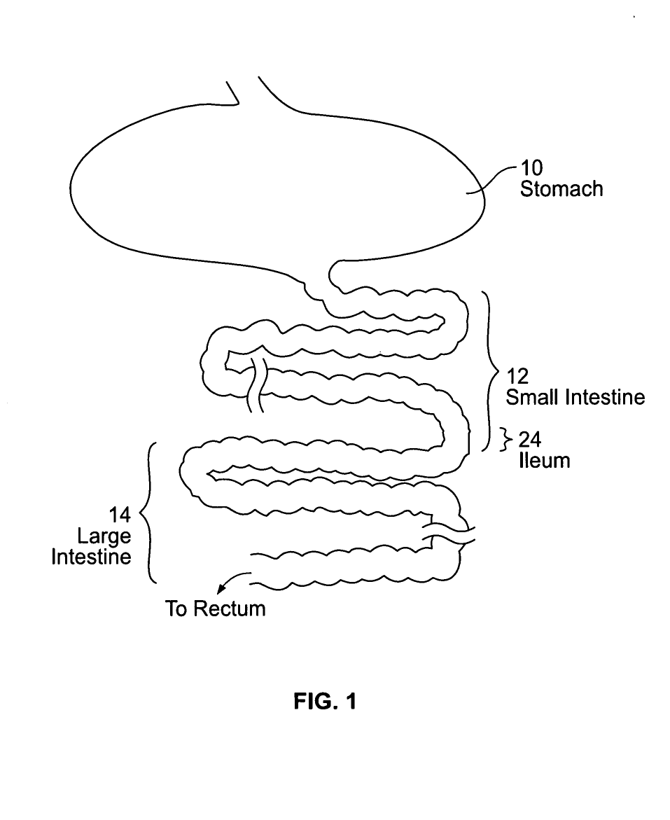

Referring now to FIG. 1, there is shown in schematic form internal human digestive tract anatomy which includes a stomach 10 which receives ingested food and the like, and which breaks down ingested substances via the host stomach gastric juices (acid, enzymes and more in an aqueous medium). Ingested materials and gastric juice so processed passes from the stomach into the small intestine 12. Ingested, broken down and macerated food osmotically passes through the walls of the small intestine to serve as human nutrients.

The large intestine 14 connects at one end to the ileum 24 of the small intestine and at the other end to the rectum. The “colon” is the vast part of the large intestine extending from the cecum to the rectum. The large intestine 14 is subject to disease conditions, e.g., infection, ulceration, colitis or cancer, among others.

Some disease conditions require removal of the large intestine after severing the ileum. Other disease conditions may be treatable, but still require severing the large intestine from the small intestine. This rest condition for the large intestine permits a period of medication, rest and hopefully repair and cure. However, during the period when the large intestine is severed from the small intestine, excrement passing from the small intestine needs to be removed from the body. Thus, whether permanently or temporarily, an ileostomy (severing the ileum to separate the small and large intestines) requires redirection of the ileum outward through the abdomen.

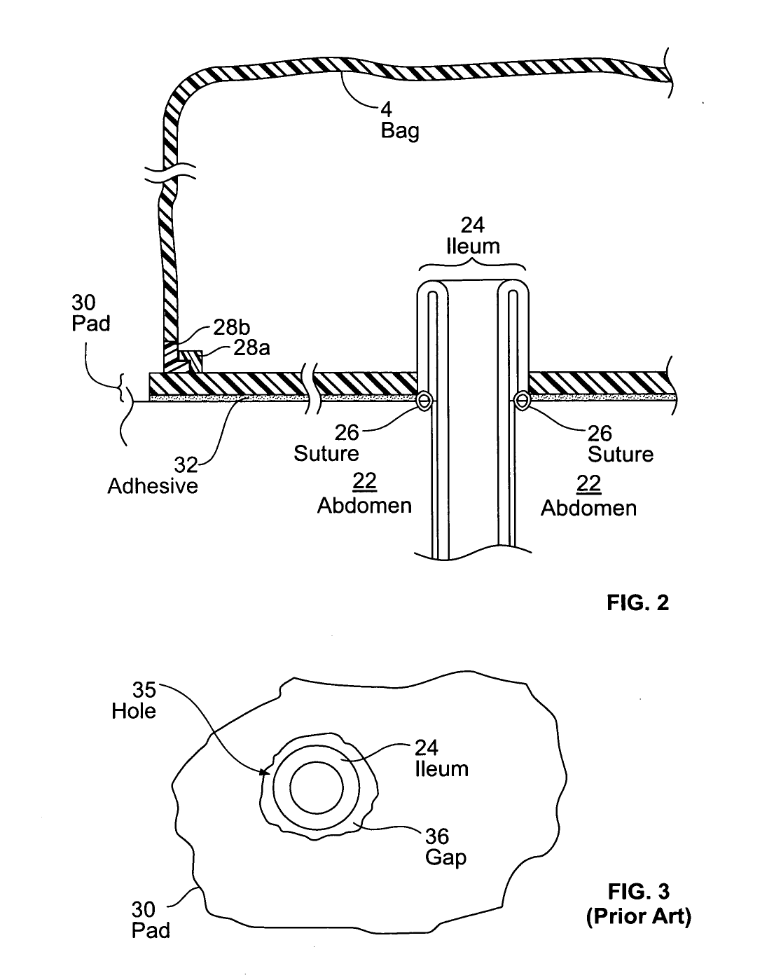

To this end, and with reference to FIG. 2, a hole (“stoma”) 23 is made in the abdomen through which the ileum 24 is passed. The ileum tube-like structure is folded upon itself and retained in place by sutures 26 connecting the ileum 24 and abdomen.

A waste impervious pad 30 having an adhesive 32 on one side is adhered to the abdomen, the pad having a central orifice 35 through which the ileum 24 passes. A waste collecting bag 40 is disposed over the ileum and sealably attached to the pad 30 via a ring 38a which mates with a ring 38b secured to the pad. That is the bag has a mating flange ring 38b which coacts with the ring 38 on the pad to retain and seal the bag 40 to the pad 30. In use, the excrement passes through the ileum 24 into the interstices of the bag 40 where it is collected. As and when appropriate, the bag 40 is separated from the ring 30 for emptying and is replaced thereon.

The adhesive pad 30 and the bag 40 are discarded from time to time (e.g., after several days) during the course of treatment or indefinitely when the large intestine is removed.

The ileum 24 can reduce in size over a course of treatment—as when the insult and inflammation from the medical procedure itself abates (size reduction). Correspondingly, the ileum 24 can increase in diameter if it becomes aggravated or inflamed.

A common difficulty with an ileostomy arises because there is a gap between the inner edge of the hole 35 roughly hand cut into the ring 30 (as by hand by scissors or the like) and the outside of the ileum 24, e.g., the gap 36 shown in FIG. 3 prior art. The materials exiting the ileum are acidic; contain enzymes; and are replete with bacteria—which can eat away at the abdomen and stoma via the gap area 36.

A patient requiring a colostomy is typically tasked with making a proper sized hole whenever the pad 30 is replaced when sent home after the procedure. A scissors is usually recommended and sometimes even provided for this purpose. A fully alert, sure-handed patient finds this a difficult task. Patients in a post-surgical, weakened condition, particularly elderly patients, find this difficult to do and thus suffer the unneeded damage resulting from leakage via such a gap 36.

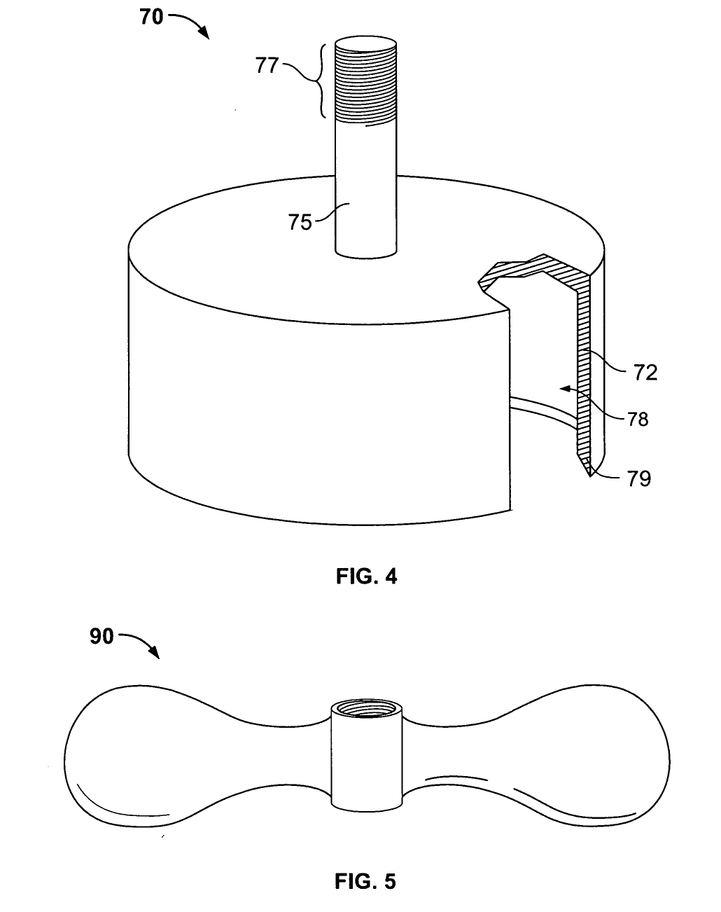

To this end, and in accordance with the principles of the present invention, the patient selectively employs an ensemble of mechanical cutting dies 70 (FIG. 4) to form a properly sized hole. Each die 70 includes a body 72 having a mounting post 75 which includes a threaded portion 77 connectable to an actuator. Each die 70 has a bottom central cylindrical portion 78 and a tapered circular cutting edge 79. Each die 70 of the ensemble has an edge ring 79 of a different size, i.e., is adapted to cut a circle of a different diameter.

A properly sized hole 35 is formed in the pad 30 by beginning with a somewhat undersized hole accomplished by selecting the corresponding die 70. The die 70 may conveniently be employed by screwing it into a stationary or hand-held punch both per se well known; or by threading an internally correspondingly threaded winged handle 90 (FIG. 5) onto the die 70 and rotating the die by hand.

Assuming the initial hole to be too small to receive its folded ileum 24, a progressively larger size die or dies 70 are employed until the proper fit is accomplished, i.e., until the hole conforms to the ileum.

This iterative procedure substantially eliminates any gap 36, leading to reduced or eliminated irritation or eating away of the abdomen about the area of hole 22 in pad 30. This also obviates any need for second or additional surgeries to move the stoma about the abdomen so that a previously attacked, inflamed area can be treated.

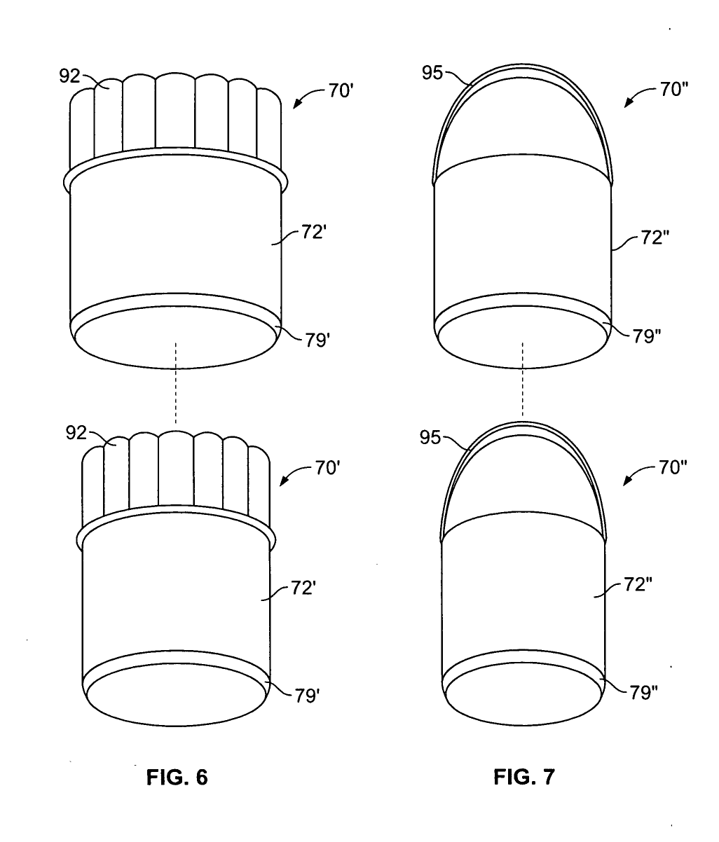

Other variations of cutting dies 70′ and 70″, per se well known in the culinary art, are shown in FIGS. 6 and 7, each having a hollow cylindrical body 72′ and 72″ and a tapered bottom cutting edge 79′ and 79″, respectively. Each die 70′ has a self contained array of partial circumferential finger grips 92. Each die 70″ has one or more handles 95 secured to the top to provide rotational and downward cutting pressure.

There are a plurality of dies 70′ (or 70″) of differing diameter to cut holes of different sizes. For storage progressively smaller dies 70′ or 70″ nest together.

While the invention has been described above using a punch or hand rotated die, other cutting tools will be readily apparent to those of ordinary skill in the art with departing from the spirit and scope of the present invention.

What is claimed is:

1. A method for preparing and applying colostomy waste removal apparatus for and upon a patient having a severed ileum extending through a stoma in the abdomen, said method utilizing a water impervious pad having first and second surfaces, an adhesive on the pad first surface, and a removably detachable waste collection vessel on the second pad surface, comprising the steps of cutting an ileum receiving hole in and through the pad, said cutting step including the steps of using a plurality of cutting tool dies of increasing size until the pad hole size conforms to the outer size of the protruding ileum, and applying the hole containing pad to the patient with the first, adhesive bearing side contacting the patient's abdomen and the ileum passing through the hole in the pad.

2. A kit for preparing and applying colostomy waste removal apparatus for and upon a patient having a severed ileum extending through a stoma in the abdomen, comprising a water impervious pad having first and second surfaces, an adhesive on said first surface of said pad, a waste collection vessel, means for removably securing said waste collection vessel to said second surface of said pad, and a plurality of cutting dies each having a hole cutting orifice edge of a different size, and means for selectively actuating a selected one of said dies.

3. A kit as in claim 2 wherein said selective actuating means comprises a punch.

4. A kit as in claim 2 wherein said selectively actuating means comprises die rotating means.
